(19) United States
(12) Patent Application Publication
(10) Pub. No.: US 2006/0213091 A1
(43) Pub. Date: Sep. 28, 2006

(54) Shoe with foot massaging sole

(76) Inventors: Walter Ometto, Padova (IT); Anna Maria Mocavero, Padova (IT)

Correspondence Address: Albert Josif;Modiano & Associati, Via Meravigli 16, Milano, 20123, IT

(21) Appl. No.: 10/565,166

(22) PCT Filed: Jul. 26, 2004
(86) PCT No.: PCT/EP04/08334

§ 371 Date: Jan. 19, 2006

(30) Foreign Application Priority Data
Aug. 1, 2003 (IT) PD2003 A 000176

(51) Int. Cl. A43B 7/06; A61F 5/14

(52) U.S. Cl. 36/141; 36/3 B

(57) ABSTRACT

A shoe comprising a sole which is provided, in its heel, with a tank in which pressurized air is accumulated by a pump actuated by the heel of the foot during normal walking. The shoe is provided with a foot supporting insole, which is provided with a plurality of holes in which a plurality of studs move at right angles to the ground; the studs are associated with an elastic chamber, which is connected to the air tank with the interposition of a discharge valve, which acts when the pressure inside the tank has reached a preset value; the studs accordingly act impulsively against the sole of the foot at preset intervals.

### TECHNICAL FIELD

The present invention relates to a shoe with foot massaging sole.

### BACKGROUND ART

It is known that the foot has nerve endings that are connected to all the organs of the body, so that by acting with localized pressures it is possible to act so as to prevent or treat disorders that in particular cause painful states.

The technique of foot treatment has been known since antiquity and has been, and still is, widely used.

When there were no drugs for treating diseases, disorders or affections of various kinds, man sought natural remedies that could provide relief.

These means were found by instinct in a manipulation of the painful regions, developing a search for points that acted by reflex on the painful region.

It was thus discovered gradually that terminations, subsequently defined as nerve endings, were concentrated in the foot and, if stimulated, could transfer the stimulus to other organs of the body.

A reflex is an involuntary but so to speak automatic response to a stimulus that causes excitation of a sensitive neuron that transmits its action up to the cerebral level.

Every part of the body is a locus of these reflexes, but more active regions are present in the foot.

FIG. 1 is a view of a sole of a foot, divided into four regions designated by the reference numerals A, B, C and D.

Region A contains the endings linked in particular to teeth, lymphatic circulation, eyes and ears; region B contains the endings linked to the gallbladder, liver, heart, bronchi, thyroid, and stomach; region C contains the endings linked to the intestine, colon, kidneys, vertebrae; and region D contains the endings linked to the genitalia, to the meniscus and to the legs.

By stimulating one of these points, it is possible to have a beneficial effect on the various organs, as demonstrated by many medical studies in reflexology and zone massage.

Shoes are known which have a foot supporting insole provided with protrusions in various regions or constituted by a mat of small studs of various hardness.

In this case, this is a passive means, which produces a sort of massage whenever, by resting the foot, the sole of the foot bears onto the protrusions or on the studs.

The action is very limited, and for some sensitive individuals resting the foot on an insole with many protrusions is unpleasant or even painful.

### DISCLOSURE OF THE INVENTION

The aim of the present invention is to provide a shoe that massages the foot actively.

Within this aim, an object of the present invention is to provide a sole in which it is possible to adjust the massaging means by choosing the points of the foot to be affected.

Another object of the invention is to provide a sole in which it is possible to adjust the intensity of the massage and also deactivate it.

A further object is to provide a sole that can be applied to any type of shoe.

A still further object is to provide a sole with active massage means that do not require energy supply sources that deplete.

Another object is to provide a massaging sole in which said massaging means also provide internal ventilation.

Another object is to provide a massaging sole whose components are structurally simple and have a low cost.

This aim and these and other objects that will become better apparent hereinafter are achieved by a shoe with foot massaging sole, characterized in that it comprises a tank, which is located preferably within the heel of the shoe sole, and a pump, which is located below the heel of the foot and is actuated by said heel of the foot while walking, said pump introducing air in said tank, which gradually increases its internal pressure, an air discharge duct branching out from said tank, an adjustable valve being interposed along said air discharge duct and opening when a preset pressure is reached, said valve feeding at least one elastic chamber provided with a plurality of studs, which are directed toward the sole of the foot and are inserted in corresponding holes provided in a foot supporting insole, said studs, when the air is discharged, protruding from said holes and acting on the sole of the foot, said outflowing air being conveyed, preferably by means of a tube, into the shoe and producing an internal ventilation.

### BRIEF DESCRIPTION OF THE DRAWINGS

Further characteristics and advantages of the present invention will become better apparent from the following detailed description of some embodiments thereof, illustrated by way of non-limiting example in the accompanying drawings, wherein:

FIG. 1 is a plan view of the sole of a foot, divided into regions of different sensitivity;

FIG. 2 is a longitudinal sectional view of the shoe according to the invention;

FIG. 3 is a sectional view of some of the parts that compose the sole;

FIG. 4 is an exploded perspective view of the parts that compose the sole;

FIG. 5 is a sectional view of a part of the sole, illustrating the studs in the inactive condition;

FIG. 6 is a sectional view of the sole of FIG. 5, in which the studs protrude from the insole due to the pressure of the air;

FIG. 7 illustrates, at the top, the chart of the increase in the air pressure in the tank and of the discharge of said air from said tank, and, at the bottom, the pulsed pressure that acts on the studs.

### WAYS OF CARRYING OUT THE INVENTION

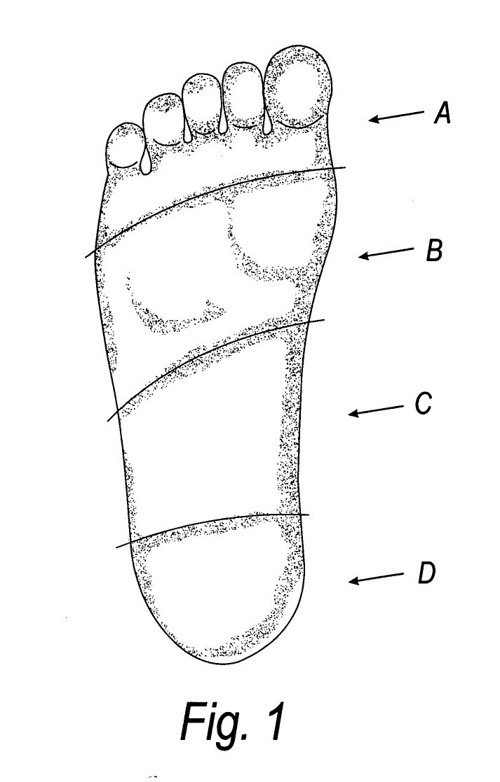

With reference to the figures cited in the introduction of the present description, FIG. 1 has already been discussed.

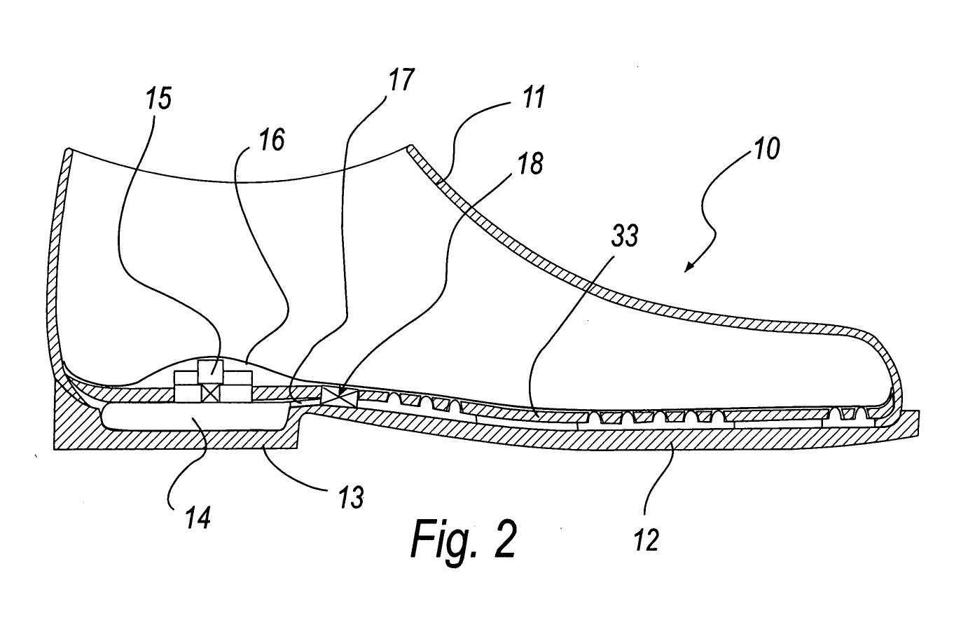

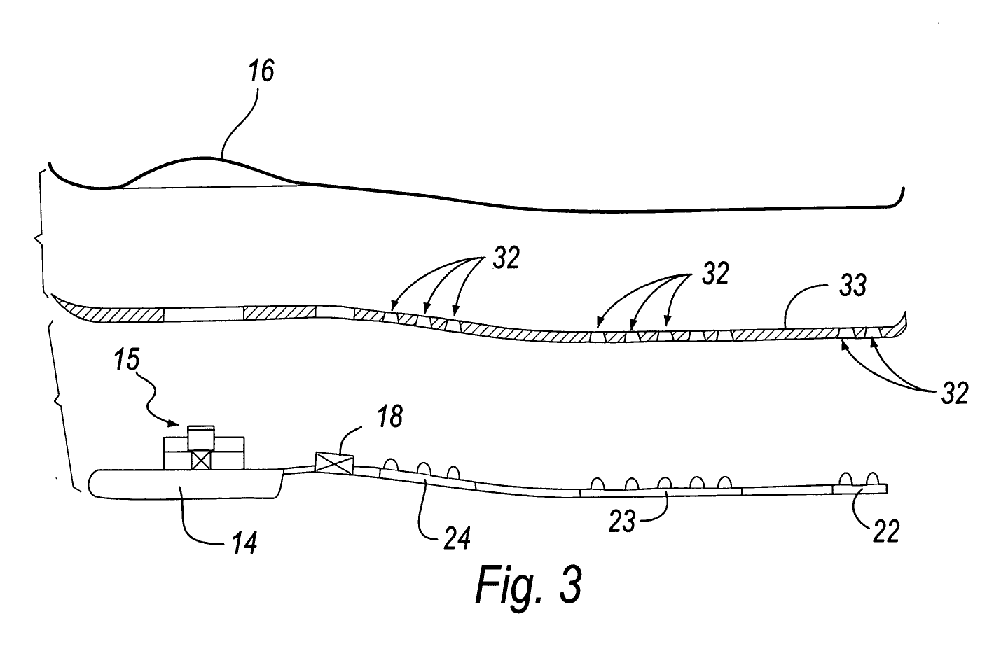

With reference to FIG. 2 and onward, a shoe, generally designated by the reference numeral 10, is composed of an upper 11, which is associated with a tread sole 12 provided with a heel 13.

A tank 14 is contained in the heel 13, and a pump 15, for example of the membrane type covered by a deformable insole 16, is provided above said tank.

Every time the pump 15 is compressed by the heel of the foot, and this occurs at each step, it introduces air in the tank 14.

Conveniently, the air can be drawn from the outside of the shoe, so as to be changed constantly.

It is in any case possible to draw the air contained in the shoe, although this does not produce air change.

The tank 14 is provided with a discharge duct 17, which is controlled by an adjustable valve 18.

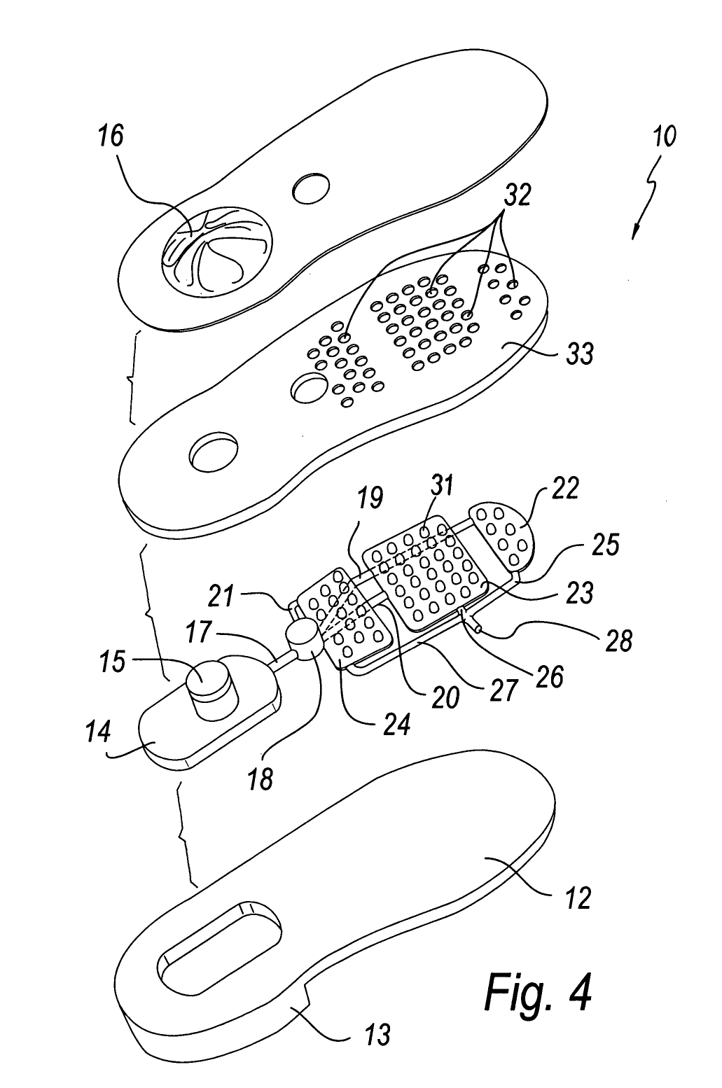

The adjustable valve 18, in the case shown in FIG. 4, has three outputs, and it is possible to select one or two or all of them by rotating for example a ring.

The valve 18 is also adjustable in terms of pressure, i.e., it opens at a chosen pressure when said pressure is reached inside the tank 14.

With reference to FIG. 4, the three outputs, designated by the reference numerals 19, 20 and 21, are connected to three elastic chambers, designated by the reference numerals 22, 23 and 24 respectively, which are located, in the case shown, at the regions A, B and C of the sole of the foot, shown in FIG. 1.

Each one of the elastic chambers 22, 23 and 24 is provided with a discharge tube, designated by the reference numerals 25, 26 and 27 respectively; said tubes merge into a single additional tube 28, which preferably leads into the inside of the shoe 10.

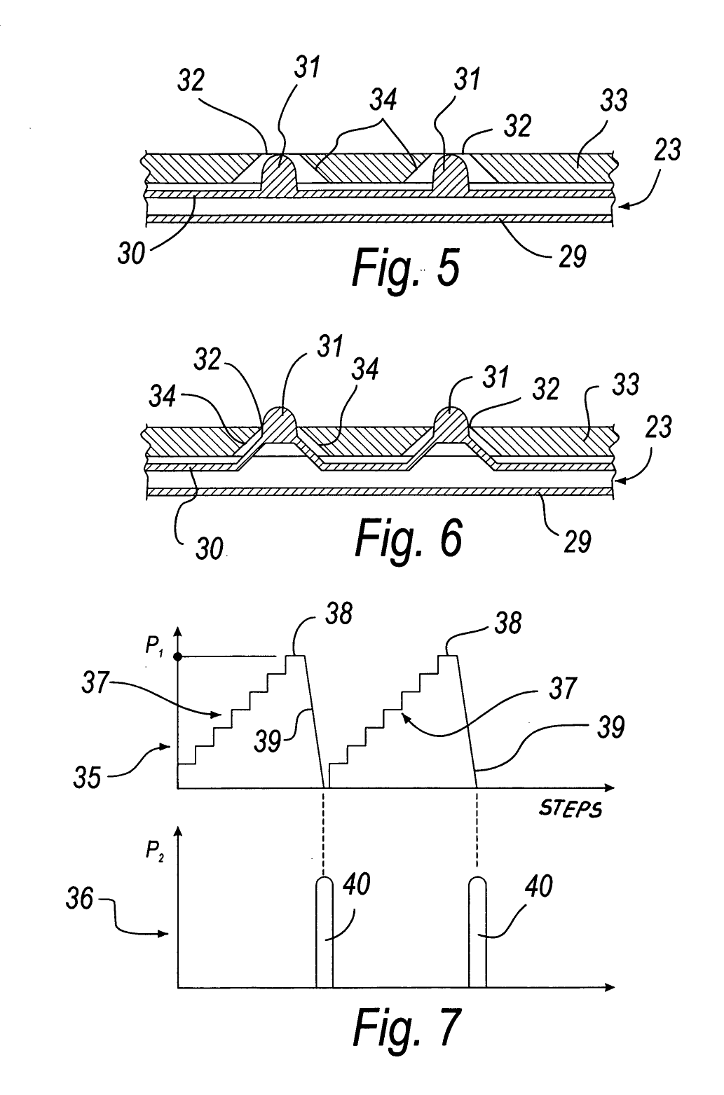

The elastic chambers 22, 23 and 24 are constituted, as shown more clearly in FIGS. 5 and 6, which illustrate by way of example the elastic chamber 23, by a bag made of an elastic material, which is provided with a lower wall 29 and with an upper wall 30.

The upper wall 30 has a plurality of studs 31 that protrude upward, i.e., toward the inside of the shoe 10.

The studs 31 are located at a plurality of holes 32, which are provided in a foot supporting insole 33 that is superimposed on the elastic chambers 22, 23 and 24.

Each hole 32 has a taper 34, with the larger end downward, which allows, as shown in FIG. 6, the elastic deformation of the upper wall 30 under the effect of the pressure of the internal air, so that the studs 31 exit from the holes 32, pressing in an impulsive manner against the sole of the foot.

FIG. 7 illustrates two charts: the upper chart 35 plots the pressure of the air inside the tank 14, and the lower chart 36 plots the pressure inside the elastic chambers 22, 23 and 24.

In the tank 14, the pressure rises in steps 37, each of which corresponds to the compression of the heel, at each stride, on the pump 15.

The impulsive discharge of the tank 14, shown by the descending ramp 39, occurs once the pressure P1, which is designated by the reference numeral 38 and is the pressure set on the valve 18, has been reached.

Correspondingly, an impulsive pressure, designated by the reference numeral 40 in the chart 36, is generated inside the elastic chambers 22, 23 and 24 and pushes the studs 31 upward and therefore against the sole of the foot in the chosen region.

In this manner, the aim is achieved of having a foot massage obtained in a manner that is active and not passive and is also adjustable.

The valve, which has been described with single-pulse discharge, can be of the sequential type, i.e., once a preset pressure is reached, the discharge occurs by means of a plurality of consecutive pulses until a low pressure that retriggers its closure is reached.

Such valve has a position in which it remains constantly open, and in this manner it is possible to exclude the foot massaging action completely.

Downstream of the valve it is possible to arrange a small tank, which contains fragrancing or sanitizing products to be replenished periodically.

These products are introduced in the shoe whenever the air that actuates the studs is discharged.

It is also possible to provide a further elastic chamber, which is arranged around the pump in the heel region, so as to also achieve massage of the part D, shown in FIG. 1.

The pump can be a multiple pump, and the tank can be divided into as many parts as there are pump sections.

In this case, with a plurality of adjustable valves, it is possible to have a different action on the various parts of the sole of the foot.

From what has been described and shown, it is evident that all the intended aim and objects have been achieved, and that in particular a sole has been obtained which allows to obtain a foot massage in the chosen manner and with an adjustable action that the wearer of the shoe can thus choose according to his sensitivity.

The combined massaging and internal ventilation action allows to achieve an optimum condition for the foot and therefore a feeling of wellbeing for the wearer of the shoe.

It is obvious that starting from the same inventive concept, the sole can be provided in various manners and with various kinds of material, depending on the quality of the shoe to be provided.

The disclosures in Italian Patent Application No. PD2003A000176 from which this application claims priority are incorporated herein by reference.

What is claimed is:

1-18. (canceled)

19. A shoe with foot massaging sole, comprising a tank, which is located within the shoe sole, and a pump, which is located below the heel of the foot and is actuated by said heel of the foot while walking, said pump introducing air in said tank, which gradually increases its internal pressure, an air discharge duct branching out from said tank, an adjustable valve being interposed along said air discharge duct and opening when a preset pressure is reached, said valve feeding at least one elastic chamber provided in an upper region, i.e., toward the sole of the foot, with a plurality of studs, which are inserted in corresponding holes provided in a foot supporting insole, said studs, when the air is discharged, protruding from said holes and acting on the sole of the foot, said outflowing air being conveyed, preferably by means of a tube, into the shoe and thus producing an internal ventilation.

20. The shoe of claim 19, wherein said pump is of the membrane type, is arranged within the heel of the shoe and is covered by a deformable insole on which the heel of the foot acts.

21. The shoe of claim 19, wherein said pump is of the piston type.

22. The shoe of claim 19, wherein said valve that controls the output duct of the tank is adjustable to a chosen pressure within a range between atmospheric pressure and the safety pressure of the tank.

23. The shoe of claim 19, wherein said valve has a single output if the sole has a single elastic chamber.

24. The shoe of claim 19, wherein said valve has a plurality of outputs, i.e., one for each one of the elastic chambers, when more than one elastic chamber is provided.

25. The shoe of claim 24, wherein in the case of a plurality of elastic chambers with said valve a single elastic chamber is selectable into which the air is discharged or a plurality of elastic chambers are selectable into which the air is discharged simultaneously.

26. The shoe of claim 24, wherein said valve discharges the air sequentially into the elastic chambers.

27. The shoe of claim 19, wherein said valve is of the type with discharge performed by a plurality of consecutive pulses.

28. The shoe of claim 19, wherein said elastic chamber is constituted by a bag made of elastic material, said bag having, on its upper wall directed toward the sole of the foot, a plurality of studs, which are inserted in corresponding holes provided in a supporting insole interposed between said elastic chamber and the sole of the foot.

29. The shoe of claim 27, wherein in the inactive condition said studs do not protrude from said foot supporting insole, while when the air pressure pulse occurs said studs protrude from said holes.

30. The shoe of claim 19, wherein said holes provided in the foot supporting insole have a frustum-like shape, with an upper end whose diameter is substantially equal to the diameter of the stud and a lower end that is much wider in order to allow the elastic deformation, under pressure, of the upper wall of the elastic chamber, with simultaneous protrusion of the studs from said foot supporting insole.

31. The shoe of claim 19, wherein in a point affected by the passage of the air during discharge, a refillable tank is provided that contains fragrancing and/or sanitizing products, which are conveyed by the air into the shoe at each discharge.

32. The shoe of claim 19, wherein said pump draws air from the outside of the shoe.

33. The shoe of claim 19, wherein said pump draws air from the inside of the shoe.

34. The shoe of claim 19, wherein the air that exits from the elastic chamber or chambers is discharged outside the shoe.

35. The shoe of claim 19, wherein said pump is composed of multiple pump, each of the individual pumps feeding a single tank, each one of said tanks being provided with an adjustable valve, each one of said valves supplying at least one elastic chamber.

36. The shoe of claim 19, wherein said elastic chamber is an elastic tube with a preset path under the foot supporting insole.
